(19) United States
(12) Patent Application Publication
(10) Pub. No.: US 2010/0040586 A1
(43) Pub. Date: Feb. 18, 2010

(54) Treatment of Injuries to the Central Nervous System

(75) Inventors: Francisco Silva, Tustin, CA (US); Luis Fidel Geffner, Guayaquil (EC)

Correspondence Address: FISH & RICHARDSON P.C., P.O. BOX 1022, MINNEAPOLIS, MN, 55440-1022, US

(73) Assignee: JUNTA DE BENEFICIENCIA DE GUAYAQUIL, Guayaquil (EC)

(21) Appl. No.: 12/543,070

(22) Filed: Aug. 18, 2009

Related U.S. Application Data
(60) Provisional application No. 61/089,765, filed on Aug. 18, 2008.

(51) Int. Cl. A61K 45/00; A61P 25/00

(52) U.S. Cl. 424/93.7

(57) ABSTRACT

The disclosure provides compositions and methods useful for treating injuries to the central nervous system (e.g., spinal cord injuries). The compositions and methods described herein can be optionally used in combination with a variety of techniques (e.g., surgical techniques) and/or therapies (e.g., physical therapy regimens) to affect treatment of injuries to the central nervous system.

### CROSS-REFERENCE TO RELATED APPLICATIONS

This application claims priority from U.S. Provisional Application No. 61/089,765, filed Aug. 18, 2008. The entire content of the prior application is incorporated herein by reference.

### BACKGROUND

Injuries to the central nervous system (e.g., spinal cord injuries) are potentially devastating disorders afflicting millions of people in the world. Such injuries can result in partial or full paralysis, incontinence, sexual dysfunction, spasticity, and sometimes extreme, chronic pain. Efficacious therapies, which treat not only the symptoms, but the injured tissue, are needed.

### SUMMARY

The disclosure relates to treating injuries to the central nervous system (e.g., spinal cord injuries). The findings described herein demonstrate that compositions containing stem cells, when administered to human patients having acute or chronic spinal cord injuries, were effective to ameliorate one or more of a variety of symptoms of their spinal cord injuries and to improve overall quality of life. As is evident from the following description, the compositions and methods described herein are useful alone or in combination with a variety of techniques (e.g., surgical techniques) and/or additional therapies (e.g., physical therapy regimens) to affect treatment of injuries to the central nervous system such as any of those described herein.

In one aspect, the disclosure features a method for treating an injury to the central nervous system. The method includes the step of delivering a therapeutically effective amount of stem cells to a subject having an injury to the central nervous system. The delivering includes: administering a first composition comprising stem cells locally to the epicenter of the injury; and administering a second composition comprising stem cells intravenously.

As used herein, “central nervous system” includes brain, brain stem, and/or the spinal cord of a subject. The term may also include the eye and optic nerve in some instances.

A “stem cell,” as used herein, refers to a cell having at least the following properties: (i) the ability to undergo multiple (e.g., two, three, four, five, six, seven, eight, nine, 10, 15, 20, or 25 or more) cell divisions while maintaining an undifferentiated state (also referred to as “self-renewal”); and (ii) the ability to differentiate into one or more (e.g., one, two, three, four, five, six, seven, eight, nine, 10, 15, or 20 or more) specialized cell types. A stem cell can be totipotent, pluripotent, multipotent, or unipotent. A totipotent stem cell can differentiate into embryonic and extra-embryonic cell types. A pluripotent stem cell can differentiate into cells derived from any of the three germ layers (e.g., ectoderm, endoderm and mesoderm). A multipotent stem cell can produce only cells of a closely related family of cells (e.g., a hematopoietic stem cell, which can differentiate into, e.g., red blood cells, white blood cells, or platelets). A unipotent stem cell can differentiate into only one cell type, but has the ability to self-renew.

In some embodiments, a stem cell can be an embryonic stem cell. In some embodiments, a stem cell can be an adult (or somatic) stem cell, which is a cell possessing the aforementioned properties and found in an organism following embryonic development. Adult stem cells can be derived from a number of tissues including, without limitation: adipose tissue, bone marrow (hematopoietic stem cells), mammary tissue, brain tissue, liver tissue, epithelium, and skin.

In some embodiments of any of the methods described herein, the stem cells delivered to the subject can be, or contain, CD34⁺ bone marrow-derived stem cells. In some embodiments, the stem cells delivered to the subject can be, or contain, embryonic stem cells and/or cord-blood derived stem cells. In some embodiments, the stem cells delivered to the subject comprise adult stem cells.

In some embodiments of any of the methods described herein, at least 1 (e.g., 1.5, 2, 2.5, 3, 3.5, 4, 4.5, 5, 5.5, 6, 6.5, 7, 7.5, 8, 8.5, 9, 10, 12, 15, 20, 30, 40, 50, 60, 70, 80, 90, or 100 or more) million stem cells/kg mass of the subject are delivered to the subject.

In some embodiments, autologous stem cells can be delivered to the subject. In some embodiments, heterologous stem cells can be delivered to the subject. In embodiments where heterologous stem cells are delivered, the methods can also include determining the blood type and/or the MHC haplotype compatibility between the donor subject and the subject to which the cells are to be delivered.

In some embodiments of any of the methods described herein, administering the first composition can include two or more (e.g., two, three, four, five, six, seven, eight, nine, 10, 11, 12, 13, 14, 15, 16, 17, 18, 19, or 20 or more) injections to the epicenter of the injury. In some embodiments, at least one of the two or more injections can be into the grey matter, or the white matter, of the epicenter.

In some embodiments, the methods can include the step of removing scar tissue from the site of injury prior to delivering the stem cells. The scar tissue can be, or contain, a glial scar.

In some embodiments, the injury can be a brain injury, a spinal cord injury, or a brain stem injury. In some embodiments, the injury can be an optic nerve injury. In some embodiments, the injury can result from, e.g., physical trauma, a cancer, an ischemic event, a developmental disorder, a neurodegenerative disorder, an inflammatory disorder, or a vascular malformation. The injury can be, e.g., acute or chronic.

In some embodiments, any of the methods described herein can include one or both of detethering the spinal cord and performing a laminectomy on the subject. The detethering and/or laminectomy can be performed prior to, or after, delivering the stem cells.

In some embodiments of any of the methods described herein, the delivering can further include administering a third composition comprising stem cells into the subarachnoid space and/or the spinal canal of the subject.

In some embodiments, any of the methods described herein can also include, prior to the delivering, culturing at least a portion of the stem cells under conditions that permit differentiation of the cells. The conditions can, e.g., permit differentiation of a plurality of the stem cells into neuronal cells.

In some embodiments, any of the methods described herein can also include subjecting the subject to a physical therapy regimen after delivering the stem cells.

In some embodiments of any of the methods described herein, the subject is a human, the injury is a spinal cord injury, and the delivering includes: administering a first composition comprising CD34⁺ bone marrow-derived stem cells locally to the epicenter of the spinal cord injury; administering a second composition comprising CD34⁺ bone marrow-derived stem cells intravenously; and administering a third composition comprising CD34⁺ bone marrow-derived stem cells into the subarachnoid space.

In some embodiments of any of the methods described herein, the subject is a human, the injury is a spinal cord injury, and the method includes, or consists of: removing scar tissue from the injured spinal cord; detethering the spinal cord; administering a first composition comprising CD34⁺ bone marrow-derived stem cells locally to the epicenter of the spinal cord injury; administering a second composition comprising CD34⁺ bone marrow-derived stem cells intravenously; and administering a third composition comprising CD34⁺ bone marrow-derived stem cells into the subarachnoid space.

In another aspect, the disclosure features a use of a composition containing stem cells for treating an injury to the central nervous system. Also featured is a use of at least two (e.g., two, three, four, five, six, seven, eight, nine, or 10 or more) compositions containing stem cells for treating an injury to the central nervous system. The composition(s) can be formulated for delivery to a subject locally to the epicenter of the injury, as an intravenous administration, or delivery to the subarachnoid or spinal canal. The compositions can be any of those described herein.

In yet another aspect, the disclosure features a use of a composition containing stem cells in the manufacture of a medicament for treating an injury to the central nervous system. Also featured is a use of at least two (e.g., two, three, four, five, six, seven, eight, nine, or 10 or more) compositions containing stem cells in the manufacture of a medicament (or medicaments) for treating an injury to the central nervous system. The medicament can be formulated for delivery to a subject locally to the epicenter of the injury, as an intravenous administration, or delivery to the subarachnoid or spinal canal. The compositions can be any of those described herein.

In another aspect, the disclosure features a composition containing stem cells for use in treating an injury to the central nervous system. The composition can also be formulated for use in conjunction with any of the surgical techniques or physical therapy methods described herein. The composition can be any of those described herein.

In yet another aspect, the disclosure features a method for evaluating the efficacy of a therapy. The method includes the steps of: collecting clinical data on the severity of an injury to the central nervous system of a subject, wherein the subject has been administered a therapy for the injury and wherein the clinical data comprises the status of the subject's urinary bladder function; assigning from a severity assessment series a severity score for the subject based on the status of the subject's urinary bladder function; optionally recording the score; and comparing the severity score to a initial severity score assigned to the subject prior to administering the therapy, wherein an increase in the severity score as compared to the initial severity score indicates that the therapy was effective for treating the injury and wherein no change, or a decrease, in the severity score as compared to the initial severity score indicates that the therapy was not effective for treating the injury. It is understood that the numeric progression of the severity series can be increasing or decreasing in order of severity. For example, in one embodiment, the severity series can be from 0 to 6, wherein 0 is lowest bladder function and 6 is complete bladder function. In this case, an numerical increase in a subject's score following treatment would indicate efficacy. In another embodiment, the severity series can be from 6 to 0, wherein 6 indicates the lowest bladder functionality and 0 is complete bladder function. In this case, a numerical decrease following treatment would indicate efficacy.

In some embodiments, the severity assessment series comprises at least six (e.g., six, seven, eight, nine, 10 or more) scores, each based on a pre-determined level of bladder functionality.

In some embodiments, the status comprises the ability of the subject to void their bladder.

In some embodiments, the therapy comprises the delivery of a composition comprising stem cells.

In another aspect, the disclosure features a network for evaluating a subject (e.g., a human patient). The network links health care providers (e.g., doctors or nurses), subjects, and an intermediary server for the purpose of providing an assessment score to the subjects. The network can be within a health care institution. The providers can be connected by a single network or can be connected by different internal networks that can communicate, e.g., using secure and/or proprietary protocols. The external network can be the internet or other well-distributed telecommunications network. Information relating to the severity of a subjects condition (e.g., an injury to the central nervous system) can be provided by the subject and/or a first provider and delivered to the another provider by way of the network. The information can include the status of the subject's urinary bladder function (including the ability to void). A second health care provider can receive the information, assign a severity assessment score, and transmit the score back to the first provider. Optionally, the subject can be directly notified.

The score can be stored/recorded in a database and/or transmitted to one or more additional health-care providers or insurers. The results can also be made available, e.g., for analysis by public health professionals and/or epidemiologists.

In another embodiment, a user (e.g., a subject or a first provider) transmits the information across a network to second provider, who receives the information and assigns an assessment score based on the information. Alternatively, a computer can process at least one parameter based on the information and assign an assessment score. The score can be transmitted back to the first provider or to the subject. The transmission can also include information useful for selecting and/or administering a therapy to the subject.

Injuries to the central nervous can result any of a variety of events including, e.g., physical trauma, a cancer, an ischemic event, a developmental disorder, a neurodegenerative disorder, an inflammatory disorder, an infection (e.g., viral infections that result in tissue, organ, or gland degeneration or injury to the central nervous system), or a vascular malformation. An injury can be acute or chronic.

Exemplary neurodegenerative disorders that can be treated using the stem cells (or compositions thereof) and methods described herein include, e.g., Parkinson's disease, Huntington's disease, amyotrophic lateral sclerosis (Lou Gehrig's disease), Rett Syndrome, certain lysosomal storage diseases (“white matter disease” or glial/demyelination disease, as described in, e.g., Folkerth ((1999) J. Neuropath. Exp. Neuro. 58), including Sanfilippo, Tay Sachs disease (beta hexosaminidase deficiency)), multiple sclerosis, amyotrophic lateral sclerosis, Parkinson's disease, progressive supra-nucleo palsy, structural lesions of the cerebellum, spinocerebellar degenerations, Friedreich's ataxia, Rufsum's disease, abetalipoprotemia, ataxia telangiectasia, Diffuse Lewy body disease, senile dementia of Lewy body type; Wernicke-Korsakoff syndrome, Creutzfeldt-Jakob disease, subacute sclerosing panencephalitis, Hallervorden-Spatz disease, cellebellar degeneration, spinobulbar atrophy, and spinal muscular dystrophy.

Ischemic events include any event that results in a restriction in blood flow to, and thus damages, the central nervous system. For example, ischemic events treatable using the methods and compositions described herein include strokes, ischemias, aneurisms, or thromboembolisms that affect the central nervous system such as the brain, brain stem, or spinal cord.

Infections that can result in an injury to the central nervous system include, e.g., meningial infections such as viral and bacterial meningitis; encephalitis, and myelitis. The infections can be caused by bacteria, viruses, parasites, fungi, or prions.

Symptoms of injuries to the central nervous system are myriad and varied depending on, e.g., the severity of the injury and the region of the central nervous system that is injured. For example, brain injuries may present different or non-overlapping symptoms with spinal injuries and vice versa. Symptoms of injuries to the central nervous system include, e.g., partial or full paralysis, incontinence, sexual dysfunction, dizziness or vertigo, depression, nausea, spasticity, difficulty breathing, loss of one or more senses (e.g., sight, hearing, taste, touch, smell), abnormal increases in blood pressure, sweating, muscle atrophy, muscle cramps, and pain.

A subject can be any vertebrate. For example, a subject can be an amphibian, a fish, a bird, a reptile, a mammal (e.g., a human, a non-human primate (e.g., ape, gorilla, macaque, chimpanzee, or lemur), a domestic animal (e.g., cat, dog, guinea pig, mouse, rat, gerbil, hamster, or rabbit), livestock (e.g., cow, pig, goat, horse, or sheep), and other non-human mammals.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention (e.g., methods for treating spinal cord injuries) will be apparent from the following detailed description and from the claims.

### BRIEF DESCRIPTION OF THE DRAWINGS

FIGS. 1A-1H are a series of photographs of magnetic resonance imaging (MRI) images of several patients at several intervals during treatment. MRI images of an acute patient (Case 1) prior to administration (FIG. 1A); at 6 months (1B); at 1 year (FIG. 1C); and approximately 2 years (FIG. 1D) after administration of bone marrow stem cells (BMSCs), demonstrate structural changes of the spinal cord as time progresses following administration of BMSCs. The images illustrate a lesion of the spinal cord at T9 from a bullet (FIG. 1A). As time progresses there is the formation of a syringomyelic cavity with spinal cord thickening and the recuperation of normal signal below the injury site (FIG. 1D). MRI images of a chronic patient (Case 5) prior to administration (FIG. 1E), at 6 months (FIG. 1F), at 1 year (FIG. 1G), and at approximately two years (FIG. 1H) after administration demonstrates structural changes of the spinal cord as time progresses following administration of BMSCs. The images illustrate a lateral hemisection of the spinal cord at T11 with residual cavities at T12.1-T12.2 (FIG. 1E). At approximately two years following BMSCs administration the MRI illustrates a decrease in the residual cavity at T12.1 (FIG. 1H). Arrows denote injury area. FIGS. 1A-1D, acute injury; FIGS. 1E-1H, chronic injury.

FIGS. 2A-2C are a series of bar graphs depicting improvements in the ASIA motor, ASIA sensory light touch, and ASIA sensory pin prick tests prior to and following treatment (at 6 months, 1 year, and 2 years after treatment). Motor improvements were observed in all cases, particularly in acute cases 1 and 4, and chronic cases 5, 6, 7, and 8 (FIG. 2A). ASIA sensory light touch and pin prick scores demonstrated that there were improvements in all cases, particular in acute cases 1 and 3 and chronic cases 5, 6, and 8 (FIGS. 2B and 2C). For case 2, the last follow up was at 1 year 6 months** and for case 7, the last follow up was at 1 year 3 months*. (ND=not done.)

FIGS. 3A and 3B are a pair of line graphs depicting a quality of life evaluation performed using the Barthel Index Score for 8 cases. Barthel scoring indicated an improvement in quality of life for all 4 acute spinal cord injury cases (FIG. 3A) and all 4 chronic spinal cord injury cases (FIG. 3B) following administration of BMSCs. A high level of improvement occurred at 6 months post-administration of BMSCs (FIGS. 3A and 3B). (ND=not done; Case 2 the last follow up was at 1 year 6 months**; while case 7 the last follow up was at 1 year 3 months*).

FIGS. 4A and 4B are a pair of line graphs depicting an evaluation of bladder function using a newly designed bladder function assessment scoring system (Geffner, Gonzalez, Santacruz, and Flor (GGSF) Bladder Function Score). There were significant improvements in bladder function for acute SCI cases 1 and 4 (FIG. 4A) and chronic SCI case 7 (FIG. 4B) after administration of BMSCs to the patients. In addition, chronic SCI cases 5 and 6 improved to complete bladder control. Overall, all SCI cases evaluated had an improvement in bladder function as assessed using the GGSF scale. ND=not done; Case 2 the last follow up was at 1 year 6 months**; while case 7 the last follow up was at 1 year 3 months*.

### DETAILED DESCRIPTION

The disclosure provides compositions and methods useful for treating injuries to the central nervous system (e.g., spinal cord injuries). The compositions and methods described herein can be optionally used in combination with a variety of techniques (e.g., surgical techniques) and/or additional therapies (e.g., physical therapy regimens) to affect treatment of injuries to the central nervous system.

### Compositions

Compositions comprising stem cells have a variety of uses such as, but not limited to, treating injuries to the central nervous system. The compositions are generally sterile and can be formulated for administration to a subject as described herein.

A stem cell can be of any vertebrate species. For example, stem cells can be from humans, non-human primates (e.g., apes, gorillas, macaques, chimpanzees, or lemurs), domestic animals (e.g., cats, dogs, guinea pigs, mice, rats, gerbils, hamsters, or rabbits), livestock (e.g., cows, pigs, goats, horses, or sheep), and other non-human mammals. Stem cells can also be from birds, reptiles, fish, or amphibians.

Methods used to identify a stem cell based on functional and/or structural characteristics. For example, a stem cell can be identified using assays to detect the presence of one or both of the above functional characteristics. One assay is the cobblestone area-forming cell (CAFC) assay, wherein a population of cells (e.g., comprising cells suspected of being stem cells) are plated on a confluent stromal cell feeder layer (see below) and monitored for ability of one or more cells to settle between the stromal cells and the substratum. (See, e.g., Bouzianas (2005) Methods in Science 25(3-4):201-210). Another assay useful for identifying stem cells based on functional characteristics is the colony forming assay. (See, e.g., Rochampally (2008) “Colony Forming Unit Assays for MSCs,” Mesenchymal Stem Cells: Methods and Protocols, Volume 449, Springer, pages 83-91 and Suzuki et al. (2002) Cell Transplantation 11(5):451-453).

Structural characteristics that can be useful to identify stem cells include, e.g., the presence of high levels of alkaline phosphatase enzyme expression or activity (Shamblott et al. (1998) Proc. Natl. Acad. Sci. USA 95:13726-13731) and/or high level expression of telomerase enzyme or telomerase activity (Odorico et al. (2001) Stem Cells 19:193-204). Stem cells can also be identified based on expression of one or more cell surface markers. For example, a stem cell can be identified as one expressing the cell surface antigen CD34 (i.e., a cell that is CD34⁺). (See the working Examples). Bone marrow-derived stem cells can be identified by any one or more of the following expression biomarkers: Stem Cell Antigen (Sca-1), CD34, c-Kit, Thy-1, CD38, and CD59.

Embryonic stem cells of human origin, e.g., are known to express a variety of cell surface markers including but not limited to stage-specific embryonic antigens 3 and 4 (SSEA-3 and SSEA-4), high molecular weight glycoproteins TRA-1-60 and RA-1-81, and alkaline phosphatase. (See, e.g., Amit M et al. (2000) Dev. Biol. 227:271-278 and Odorico et al., supra). In their undifferentiated state, the embryonic stem cells retain their ability to express the transcription factor October 4 (Oct4). (See, e.g., Reubinoff et al. (2000) Nature Biotechnology 18:399-404.; Schuldiner et al. (2000) Proc. Natl. Acad. Sci. USA 97:11307-11312).

Methods for detecting the mRNA or protein expression of cell surface markers or cytoplasmic proteins (e.g., telomerase or transcriptions factors such as Oct4) are known in the art and exemplified in the working Examples. (See also, e.g., Sambrook et al., Molecular Cloning: A Laboratory Manual, Cold Springs Harbor Laboratory, New York (1989, 1992) and Ausubel et al., Current Protocols in Molecular Biology, John Wiley and Sons, Baltimore, Maryland (1989)). For example, protein expression by a cell can be detected using western blot analysis or antibody-coupled fluorescence-assisted cell sorting (FACS).

Generally, stem cells for use in the compositions and methods described herein can be obtained from many different sources. Methods for obtaining stem cells vary widely based on the particular type of stem cell and the tissue from which a stem cell is to be isolated. However, such methods are both known in the art and exemplified in the working Examples.

For example, bone marrow-derived stem cells can be obtained from donor bone (e.g., the iliac bone) by aspiration using, e.g., a multi-holed needle. The isolated bone marrow can be subjected to centrifugation to obtain a plasma fraction and a “buffy coat layer” fraction, the latter containing stem cells and other white blood cells. Stem cells can be further enriched by subjecting the buffy coat layer to filtration using, e.g., ficol-hypaque with heparin and centrifugation. Optionally, the supernatant can be washed and a sample of the supernatant processed (e.g., by FACS analysis) to determine the number of CD34⁺/CD45⁻ cells in the enriched mixture. The stem cells can be further purified based on expression of CD34.

Embryonic stem cells can be isolated from the primordial germinal ridge of the developing embryo and from non-embryonic tissues, including, without limitation, bone marrow, brain, liver, pancreas, peripheral blood, placenta, skeletal muscle, and umbilical cord blood. Embryonic stem cells can be isolated from the inner cell mass of blastocyst-stage embryos (See, e.g., Odorico et al., supra; Thomson et al. (1995) Proc. Natl. Acad. Sci. USA. 92:7844-7848.; Thomson et al. (1998) Science 282:1145-1147).

Adipose-derived stem cells can be isolated from fat deposits, e.g., using liposuction techniques (e.g., tumescent, ultrasonic-assisted, or power-assisted liposuction). Stem cells can be released from fat deposit tissue using a protease such as a collagenase. Methods for isolation of stem cells from fat have also been described in U.S. Patent Publication No. 20050153442, the contents of which are incorporated by reference in their entirety.

Stem cells can also be isolated from liver tissue or brain tissue. For example, liver tissue can be obtained by biopsy or surgical excision by perfusion. Methods for isolating stem cells from brain tissue are described in, e.g., U.S. Pat. Nos. 5,851,832 and 5,968,829, the contents of each of which are incorporated by reference in their entirety. Stem cells can be isolated from peripheral blood using standard phlebotomy techniques.

In some embodiments, isolated stem cells can be cultured under conditions that permit the differentiation of a plurality of the cells. As used herein, a “plurality” of a population refers to more than one (1) in the population. Thus, a plurality of cells is more than one cell in a population. A plurality of cells can be, e.g., greater than (or at least) 2 (e.g., 3, 4, 5, 6, 7, 8, 9, 10, 12, 15, 17, 20, 22, 25, 27, 30, 32, 35, 37, 40, 42, 45, 47, 50, 52, 55, 60, 62, 65, 70, 72, 74, 77, 80, 82, 85, 87, 90, 92, 95, or 97 or more) % of the cells in a population.

The conditions can, e.g., permit the differentiation of a plurality of cells into a single, specialized cell type or more than one (e.g., two, three, four, five, six, seven, eight, nine, or 10 or more) different, specialized cell type. For example, adipose-derived stem cells can be cultured under conditions that permit differentiation of cells into one or more of bone, fat, muscle, cartilage, and neurons. (See, e.g., Gimble (2003) Expert Opin. Biol. Ther. 3(5):705-13; Fujimura et al. (2005) Biochem. Biophys. Res. Commun. 333(1):116-21; and Kingham et al. (2007) Exp. Neurol. 207(2):267-74). Bone marrow-derived stem cells can be cultured under conditions that permit differentiation of the cells into one or more of muscle cells, skin, liver, lung, epithelial cells, neurons, and glial cells. For example, cultures of bone marrow-derived stem cells can be induced to form neurons by culturing the cells in media containing DMEM, 2% dimethylsulfoxide (DMSO), and 200 mM butylated hydroxyanisole (BHA). (See, e.g., Crain et al. (2005) J. Neurol. Sci. 233(1-2):121-3; Woodbury et al. (2000) J. Neurosci. Res. 61:364-370; and Deng et al. (2001) Biochem Biophys Res Commun. 282:148-182).

Embryonic stem cells may be induced to undergo lineage-specific differentiation in response to a variety of cytokines. Retinoic acid, basic fibroblast growth factor, bone morphogenetic protein 4, and epidermal growth factor induce differentiation of embryonic stem cells into both ectodermal (skin, brain) and mesodermal (chondrocyte, hematopoietic) lineages (Schuldiner et al., supra). Other factors, such as nerve growth factor and hepatic growth factor, promote differentiation along all three embryonic lineages (ectodermal, endodermal, and mesodermal). (See, e.g., Itskovitz-Eldor et al. (2000) Mol. Med. 6:88-95 and Reubinoff et al., supra). Other factors such as platelet derived growth factor can also promote glial cell differentiation (Brustle et al. (1999) Science 285(5428):754-6).

In some embodiments, stem cells can be cultured under conditions that allow the differentiation of the cells into one or more certain specialized subtypes, but inhibit the differentiation of the cells into other specialized subtypes. For example, transforming growth factor beta 1 (TGF-β1) and activin A inhibit endodermal and ectodermal differentiation of embryonic stem cells, while promoting differentiation of stem cells into skeletal and cardiac muscle (Schuldiner, supra).

It is understood that compositions containing one or more differentiated cell types (e.g., compositions in which a portion of the stem cells were subjected to conditions that permit cell differentiation) will also contain stem cells in an amount effective to treat a subject's injury to the central nervous system. The portion of stem cells can be, e.g., 1 (e.g., 2, 5, 10, 15, 20, 25, 30, 35, 40, 45, 50, 55, 60, 65, 70, 75, 80, 85, 90, or 95 or more) % of the total stem cells in the composition so long as the remaining number of stem cells in the composition is an effective amount in accordance with the disclosure.

In some embodiments, the compositions can contain stem cells that contain a recombinant nucleic acid (e.g., an expression vector). Such nucleic acids can be used to express any of a variety of therapeutic proteins (e.g., any of the growth factors or angiogenic factors described herein). Methods for generating an expression vector encoding a therapeutic protein, as well as methods for introducing the nucleic acid into a cell, are known in the art. (See, e.g., Sambrook et al., supra). For example, transfection of mammalian cells can feature, for example, the introduction of a vector to the cells using calcium phosphate, electroporation, heat shock, liposomes, or transfection reagents such as FUGENE® or LIPOFECTAMINE®, or by contacting naked nucleic acid vectors with the cells in solution (see, e.g., Sambrook et al., supra).

Wherever possible, any stem cell culture conditions should be optimized to prevent, or minimize the likelihood, that the stem cells undergo malignant transformation and thus form tumors when delivered to a subject. For example, a growth medium comprising glucose, insulin, transferrin, T₃, fetal calf serum, and tissue extracts has been show to allow stem cells derived from liver to grow without malignant transformation. (See, e.g., Rogler (1997) Am. J. Pathol. 150:591; Alison (1998) Current Opin. Cell Biol. 10:710; and Lazaro et al. (1998) Cancer Res. 58:514).

Some methods for the isolation and maintenance of stem cells rely on the use of embryonic fibroblasts such as mouse embryonic fibroblasts (MEF). Prior to culture, the embryonic fibroblasts are irradiated to reduce cell proliferation without compromising metabolic function (Shamblott et al. 1998, Proc Natl Acad Sci USA 95:13726-13731; Amit et al 2000, Dev Biol 227:271-278). Isolated stem cells are then plated onto the irradiated embryonic cell feeder layer culture (Reubinoff et al., supra and Thomson et al., supra).

Methods of culturing stem cells without an embryonic cell feeder layer can be found in, e.g., U.S. Patent Publication Nos. 20030152558 and 20020090723, the contents of each of which are incorporated herein by reference in their entirety.

In some embodiments, the compositions contain totipotent stem cells, pluripotent stem cells, and/or multipotent stem cells. In some embodiments, the compositions contain at least one type of stem cell capable of differentiating in vitro or in vivo into a neuronal cell such, e.g., a basket cell, a Betz cell, a Purkinje cell, a pyramidal cell, a Renshaw cell, or a motor neuron).

In some embodiments, a composition can contain one type of stem cell. In some embodiments, a composition can contain two or more (e.g., three, four, five, six, seven, eight, nine, or 10 or more) different types of stem cells. For example, a composition can contain bone marrow-derived stem cells or a composition can contain a combination of bone marrow-derived stem cells, neural stem cells, and embryonic stem cells.

In some embodiments, a composition can contain other cells in addition to stem cells. For example, a composition can contain stem cells and one or more of astrocytes, oligodendrocytes, and neurons.

In some embodiments, the percentage of stem cells in a population of cells can be, e.g., at least (or greater than) 5 (e.g., 6, 7, 8, 9, 10, 12, 15, 17, 20, 22, 25, 27, 30, 32, 35, 37, 40, 42, 45, 47, 50, 52, 55, 60, 62, 65, 70, 72, 74, 77, 80, 82, 85, 87, 90, 92, 95, or 97 or more) % in a composition.

In some embodiments, a composition can contain greater than (or at least) 1×10⁵ (e.g., 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, or 9×10⁸ or more) stem cells/kg weight of the subject to which the cells are to be delivered. In some embodiments, the composition can contain between 500,000 and 2,000,000 (e.g., 500,000 to 1,000,000; 500,000 to 750,000; 750,000 to 1,000,000; 750,000 to 2,000,000; 750,000 to 1,500,000; 1,000,000 to 2,000,000; 1,000,000 to 1,500,000; or 1,500,000 to 2,000,000) stem cells/kg weight of the subject. The composition can include e.g., greater than (or at least) 1×10⁶ (e.g., 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 1×10⁹, 2×10⁹, 3×10⁹, 4×10⁹, 5×10⁹, 6×10⁹, 7×10⁹, 8×10⁹, or 9×10⁹ or more) total cells/kg weight of the subject.

The composition can be formulated to include one or more stem cell growth factor agents that stimulate the growth of, or enhance the viability of, a stem cell. Such agents include, e.g., human growth hormone (HGH), testosterone, brain derived neurotrophic factor (BDNF), estrogen, pregnenolone, dehydroepiandrosterone (DHEA), granulocyte colony stimulating factor (G-CSF), granulocyte macrophage colony stimulating factor (GM-CSF), parathyroid (synthetic or natural) hormone, erythropoietin (EPO), stem cell factor (SCF), and leukemia inhibitory factor (LIF).

Prior to, or after, formulation of the compositions, the stem cells can be frozen at liquid nitrogen temperatures and stored for long periods of time. The cells can be stored in, or the compositions can thus include, any of a variety of cryoprotective agents (e.g., glycerol or dimethylsulfoxide (DMSO)) that aid in the preservation of cells at low temperatures. Where the stem cells are to be frozen, the compositions are generally formulated such that subsequently thawed cells retain activity (e.g., self-renewal and potency). A pharmaceutical composition can also be suitably formulated for refrigeration (e.g., storage at 2-8° C.).

### Pharmaceutical Compositions

Any of the stem cell compositions described herein can be formulated as pharmaceutical compositions. Suitable pharmaceutical compositions are known in the art and exemplified in the working Examples.

Generally, a pharmaceutical composition includes a pharmaceutically acceptable carrier, additive, or excipient and is formulated for an intended mode of delivery, e.g., intraperiteneal, intravenous, or intramuscular administration, direct injection into a tissue of the central nervous system (e.g., brain tissue, spinal tissue, or brain stem), or any other route of administration described herein. For example, a pharmaceutical composition for intravenous administration can include a physiological solution, such as physiological saline and water, Ringers Lactate, dextrose in water, Hanks Balanced Salt Solution (HBSS), Isolyte S, phosphate buffered saline (PBS), or serum free cell media (e.g., RPMI). The compositions can also include, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of a composition can be adjusted with acids or bases, such as hydrochloric acid or sodium hydroxide.

In some embodiments, the pharmaceutical composition can include blood plasma (e.g., blood plasma from a subject to be treated with the composition).

Pharmaceutical compositions should be stable under the conditions of processing and storage and must be preserved against potential contamination by microorganisms such as bacteria and fungi. Prevention of contamination by microorganisms can be achieved by various antibacterial and antifungal agents, e.g., antibiotics such as aminoglycosides (e.g., kanamycin, neomycin, streptomycin, and gentamicin), ansaycins, and quinalones.

The pharmaceutical composition can be formulated to include one or more additional therapeutic agents. For example, a composition can be formulated to include one or more growth factors (e.g., an angiogenic factor or a neural growth factor) and/or one or more anti-inflammatory agents. Angiogenic factors include any agent capable of stimulating the growth of new blood vessels (e.g., spontaneous blood vessel formation or blood vessel formation from a pre-existing vessel). Angiogenic factors include, e.g., fibroblast growth factor (FGF), vascular endothelial growth factor (VEGF), platelet derived growth factor (PDGF), transforming growth factor beta (TGF-β), angiopoietins (e.g., Ang1 and Ang2), matrix metalloproteinases (MMPs), and DII4. Neural growth factors include any agent capable of stimulating the growth of, promoting the survival of, or stimulates the differentiation of a cell into, neurons. Such neural growth factors include, e.g., nerve growth factor (NGF), basic fibroblast growth factor (bFGF), epidermal growth factor (EGF), fibroblast growth factor 2 (FGF-2), brain derived neurotrophic factor (BDNF), and insulin-like growth factor-I (IGF-I). (See, e.g., Arsenijevic et al. (2001) J. Neurosci 21(18):7194-7202).

In some embodiments, any of the pharmaceutical compositions described herein can be included in a container (e.g., a blood storage bag), pack, or dispenser (e.g., a syringe) together with instructions for delivery as described, e.g., in the following sections and the working Examples.

### Methods for Treatment

The compositions described herein are useful for, inter alia, treating a subject having an injury to the central nervous system such as any of those described herein. The compositions can be delivered to a subject in various ways as appropriate to deliver stem cells to the central nervous system including, but not limited to: parenteral (including intravenous and intraarterial administration), intrathecal administration, intraventricular administration, intraparenchymal, intracranial, intracisternal, intrastriatal, intranigral administration, or any other route described herein or suitable for direct delivery to a the central nervous system of a subject. In addition, a combination of two or more of any of these routes can be used to deliver stem cells. For example, stem cells can be delivered to a subject intravenously and by direct injection into the site of injury (e.g., spinal cord injury). In another example, stem cells can be delivered to a subject intravenously, directly injected in the site of injury, and injected into the subarachnoid space. In some embodiments, the stem cells can be administered by way of lumbar puncture.

The compositions described herein are dosed and delivered in accordance with good medical practice, taking into account, e.g., the clinical condition of the subject; the site and method of administration; scheduling of administration; the subject's age, sex, race, and body weight; other medications that the subject has taken or is currently taking; and other factors known to medical practitioners.

Delivery of stem cells can be accomplished using techniques well known in the art as well as those described herein and exemplified in the working Examples. For example, the working Examples describe methods for delivering stem cells to various sites within the spinal cord of a human subject as well intravenous delivery of stem cells.

The stem cells and compositions thereof can be, in some embodiments, injected into one or more of a number of sites including, e.g., the epicenter of the injury, the subarachnoid space, the spinal canal, and the syringomyelic cavities. Specific sites of injection can be, e.g., portions of the gray matter or white matter. Stem cells can be injected into one or more (e.g., one, two, three, four, five, six, seven, eight, nine, or 10 or more) different sites by one or more (e.g., two, three, four, five, six, seven, eight, nine, 10, 12, or 15 or more) injections at each site. For example, stem cells can be administered to the epicenter of an injury to a subject's spinal cord through a series of two or more injections. In another example, stem cells can be delivered to a subject by two or more injections into the injury epicenter and two or more injections into the subarachnoid space. In some embodiments, any of the aforementioned delivery methods can be combined with intravenous delivery of stem cells. For example, stem cells can be delivered to a subject by intravenously administration along with two or more injections of stem cells into the injury epicenter and two or more injections of stem cells into the subarachnoid space.

Methods for determining the site, or epicenter, of an injury to the central nervous system are known in the art and described in the working Examples. Magnetic Resonance Imaging (MRI) is one method for determining the site of injury. For example, MRI can be used to identify the particular segmental spinal cord level (e.g., C1-6, C7, T1-T6, T7-L1, L2, L3, L4, or L5) that is affected by the injury. (“C” refers to cervical vertebrae, “T” refers to thoracic vertebrae, and “L” refers to lumbar vertebrae, and the numbering refers to the vertebrae number in the series; see, e.g., Flanders et al. (1999) Am. J. Neuroradiol. 20:926-934).

In some embodiments, the methods include the step of administering (e.g., by injection) a composition comprising stem cells to the epicenter of an injury to the central nervous system. In some embodiments, the methods include the step of delivering stem cells to a subject, wherein the delivery includes: (i) administering (e.g., by injection) a first composition comprising stem cells to the epicenter of an injury to the central nervous system and (ii) intravenously administering to the subject a second composition comprising stem cells. In some embodiments, the methods can include the step of: (i) administering (e.g., by injection) a first composition comprising stem cells to the epicenter of an injury to the central nervous system; (ii) intravenously administering to the subject a second composition comprising stem cells; and (iii) administering a third composition comprising stem cells to the subarachnoid space, spinal canal, and/or syringomyelic cavities.

Each of the first, second, and third compositions can be portions of the same composition. For example, one composition (such as a composition containing a population of bone marrow-derived stem cells) can be partitioned into three portions. The three portions can be equal portions or can be of different proportion (e.g., different liquid volumes or different numbers of stem cells). Alternatively, the first, second, and third compositions can be different compositions. For example, each of the compositions can be formulated differently based on the route of administration. In some embodiments, two of the compositions can be the same and a third, different. For example, the first and third composition can be the same composition (or a portion of the same composition) and the second composition can be a different composition (e.g., different formulation or containing different stem cells).

The first, second, and third compositions can, in some embodiments, contain the same type of stem cell or mixtures of the same types of stem cells. For example, each of the first, second, and third compositions can contain bone-marrow derived stem cells. In some embodiments, one or more of the first, second, and third compositions can each contain different types of stem cells. For example, the first and third compositions can contain bone-marrow derived stem cells and the second composition can contain embryonic stem cells.

In some embodiments, more than three (e.g., four, five, six, seven, or eight or more) compositions containing stem cells are administered to the subject. It is understood that each of the three or more compositions can be the same or different from one another, as elaborated on above.

In some embodiments, between 500,000 and 2,000,000 (e.g., 500,000 to 1,000,000; 500,000 to 750,000; 750,000 to 1,000,000; 750,000 to 2,000,000; 750,000 to 1,500,000; 1,000,000 to 2,000,000; 1,000,000 to 1,500,000; or 1,500,000 to 2,000,000) stem cells/kg weight of the subject can be delivered to the subject in total. In some embodiments, about 1.2×10⁶ stem cells/kg weight of the subject are delivered to the subject.

In some embodiments, between 500,000 and 500,000,000 (e.g., 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷,7×10⁶, 8×10⁷, 9×10⁷, 1×10⁸, 2×10⁸, 3×10⁸, 4×10⁸, or 5×10⁸) stem cells/kg weight of the subject can be delivered to the subject in total.

In some embodiments, stem cells are delivered to the subject only once. In some embodiments, multiple (e.g., two, three, four, five, six, seven, eight, nine, 10, 11, 12, 13, 14, 15, or 20 or more) deliveries are made. For example, multiple deliveries of stem cells can be made over the course of several (e.g., two, three, four, five, six, seven, eight, nine, 10, 14, 21, 28, or 31 or more) consecutive days (e.g., one delivery each day for seven days or one delivery every other day for seven days). Stem cells can be delivered to a subject for several months (e.g., one delivery per month for six months, or one delivery per week for two months).

Stem cells can be delivered to a subject at various time points after injury. For example, the cells can be delivered immediately following an injury (e.g., from 1 to 8 (e.g., 1.5, 2, 2.5, 3, 3.5, 4, 4.5, 5, 5.5, 6, 6.5, 7, 7.5, or 8) hours after the injury occurs). The cells can be delivered to a subject less than 10 (e.g., 9, 8, 7, 6, 5, 4, 3, 2, or 1) days after an injury occurs. The cells can be delivered to a subject less than 6 (e.g., 5, 4, 3, 2, or 1) weeks after an injury occurs. In some embodiments, stem cells can be delivered to a subject up to 10 years (e.g., 9, 8, 7, 6, 5, 4, 3, 2, or 1) years after an injury occurs. The compositions and methods described herein can be used at any time following an injury or during the course of a chronic injury.

It is understood that regardless of the site, combination of sites, route of administration, combination of route, a therapeutically effective amount of stem cells (or a composition comprising the stem cells) is delivered to the subject. As used herein, an “effective amount” or “therapeutically effective amount” of a composition or stem cells is the amount that is sufficient to provide a beneficial effect to the subject to which the composition or cells are delivered. The effective amount can be the amount effective to achieve an improved survival rate, a more rapid recovery, an improvement in the quality of life, or an improvement or elimination of one or more symptoms associated with a subject's condition (e.g., an injury to the central nervous system).

The efficacy of a given treatment in treating an injury to the CNS (e.g., a spinal cord injury) can be defined as an improvement of one or more symptoms of the injury (e.g., any of the symptoms described above) by at least 5% (e.g., at least 10%, at least 15%, at least 20%, at least 25%, at least 30%, at least 40%, at least 50%, at least 55%, at least 60%, at least 65% or more). In some cases, efficacy of a stem cell treatment can be determined from the stabilization of one or more worsening symptoms associated with the injury (i.e., the treatments curtail the worsening of one or more symptoms of the injury).

Methods for determining a therapeutically effective amount of a composition comprising stem cells, or a therapeutically effective amount of stem cells, are known in the art and exemplified in the working Examples. Clinical indicia by which efficacy can be measured include, e.g., restoration of nerve impulse conduction, an increase in conduction action potential, and magnetic resonance imaging to detect morphological changes in the spinal cord. Motor and sensory functions can be evaluated before and after stem cell administration using the American Spinal Cord Injury Association (ASIA), Frankel, and Ashworth scoring systems. (See, e.g., Sykova et al. (2006) Cell Transplant 15:675-87; Katz et al. (1997) PM&R Secrets. Philadelphia; Hanley & Belfus, page 487; and the working Examples). Overall quality of the life can be evaluated through the use of the Barthel Index system. (See, e.g., Mahoney et al. (1965) Maryland State Medical Journal 14:56-61 and the working Examples). Clinical indicia also include improvements in patients urinary bladder function following administration of stem cells. For example, the working Examples describe a clinical scoring system that takes in account patient bladder functionality including sensation and ability to control voiding. (See working Examples). It is understood that efficacy of a treatment in a subject can be evaluated using one or a combination or two or more of any of the foregoing methods.

In some embodiments, the methods and compositions described herein are able to induce remyelination and/or nerve regeneration in a subject. Thus, efficacy of a treatment can be evaluated as an increase in myelination in a subject following stem cell administration. Methods for monitoring a subject for an increase in myelination or nerve regeneration include both quantitative and qualitative techniques. For example, nerve regeneration or remyelination in a subject can be determined by evaluating the nerve tissue of a subject before and after treatment using, e.g., Magnetic Resonance Imaging (MRI) scans, Positron-Emission Tomography (PET) scans, Diffusion-Weighted Imaging (DW-I, or DW-MRI), Diffusion Tensor Imaging, Myelography, Magnetization Transfer. Remyelination following a stem cell treatment can be evaluated as an increase in white matter volume (e.g., nerve mass of the spine or brain). In some instances, the extent or occurrence of remyelination in a subject can be assessed by directly measuring the thickness of myelin in a subject using, e.g., magnetic resonance spectroscopy scans. Also, remyelination or nerve regeneration in a subject could be measured as an increase in the speed of transmission of a signal from the ears, eyes, or skin to the brain, as determined through evoked potential testing. Qualitative techniques include, e.g., semi-quantitative or qualitative assessments of their neuropsychology (e.g., the status of various abilities such as memory, arithmetic, attention, judgment and reasoning) or symptoms (clinical parameters) presented by the subject including, e.g., any of the symptoms of injuries to the CNS described above. It is understood that all of the above testing methods can be used generally to evaluate restoration in nerve cell function following treatment.

Effective amounts of stem cells can include, e.g., between 500,000 and 2,000,000 (e.g., 500,000 to 1,000,000; 500,000 to 750,000; 750,000 to 1,000,000; 750,000 to 2,000,000; 750,000 to 1,500,000; 1,000,000 to 2,000,000; 1,000,000 to 1,500,000; or 1,500,000 to 2,000,000) stem cells/kg weight of the subject. In some embodiments, approximately 1.2×10⁶ stem cells/kg weight of the subject can be therapeutically effective.

In some embodiments, the stem cells are “autologous” (i.e., the donor and the recipient of the stem cells are the same subject). Where stem cells are obtained from another subject of the same species as the subject (“allogeneic” cells), it is preferable to determine blood type or MHC haplotype compatibility between the donor subject and the subject to which the cells are to be delivered, prior to the delivery. Suitable methods for determining blood type and/or MHC haplotype compatibility are known in the art. (See, e.g., Petersdorf et al. (2007) PLoS Med. 4(1):e8).

As is evident from the working Examples, the stem cells can be delivered in conjunction with a variety of surgical techniques and/or additional therapies. For example, prior to delivering the stem cells to the subject, a laminectomy can be performed on the subject, wherein the lamina is removed to increase the available space for neural tissues in the affected area. Briefly, a small incision is made in the back of the subject and an opening into the spinal is slowly created. The process pushes the muscles out of the way instead of having to cut or tear them. The methods can also include removing scar tissue from the site of injury to the central nervous system. The scar tissue can be, or contain, a glial scar or other fibrotic scar tissue.

The methods can also include, prior to delivering the stem cells, exposing the dura mater, the tough and rigid outermost layer of the meninges surrounding the brain and spinal cord. In some embodiments, the scar tissue is removed after the dura mater is exposed.

In some embodiments, the methods can include decompression of the spinal cord, e.g., detethering the spinal cord.

The above surgical techniques, as well as methods for performing the techniques, are known in the art and are described in the working Examples. (See also, e.g., Haher et al. (2003) “Surgical Techniques for the Spine”; Crock (1983) “A Short Practice of Spinal Surgery,” 2^{nd} edition, Springer-Verlag Wien New York; and Benzel (1999) “Spine Surgery Volume 1: Techniques, Complication Avoidance, and Management,” Churchill Livingstone).

In some embodiments, stem cells are delivered to the brain of a subject (e.g., ischemic brain, injured brain, injured spinal cord, and into brain that exhibits symptoms of stroke). Methods for delivering cells to the brain are described in, e.g., Bjorklund and Stenevi (1985) Neural Grafting in the Mammalian CNS, eds. Elsevier, pages 169-178, the contents of which are incorporated by reference. Briefly, brain surgery is generally aided by computed axial tomography (CAT) scan to establish, e.g., the coordinates of the region of the brain to which the cells should be administered. A small hole is the subject's skull can be made by use of a drill and the dura is also pierced. The cells can be injected into the brain to the correct coordinates by way of a needle (e.g., an infusion cannula). The subject can be examined by CAT scan postoperatively for hemorrhage or edema.

Administration of the cells or compositions to a subject can also be performed in combination with anti-inflammatory agents, growth factors, and/or angiogenic factors. The cells can also be cultured, e.g., with any of the growth factors (e.g., nerve growth factor (NGF)) prior to administration to the subject. The cells can also be cultured in any neuronal differentiation medium prior to administration to the subject (as described herein).

Stems cells or pharmaceutical compositions thereof described herein can be administered to a subject as a combination therapy with another treatment, e.g., a treatment for an injury to the central nervous system (CNS). For example, the combination therapy can include administering to the subject (e.g., a human) one or more additional agents that provide a therapeutic benefit to the subject who has an injury to the CNS. Additional therapeutic agents include, e.g., growth factors (e.g., an angiogenic factor or a neural growth factor) and/or anti-inflammatory agents such as any of those described above. The additional therapeutic agents can also be, e.g., a steroid (e.g., methylprednisolone). Anti-inflammatory agents include, e.g., non-steroidal anti-inflammatory drug (NSAID; e.g., salicylates (e.g., aspirin) or COX-2 inhibitors), a disease-modifying anti-rheumatic drug (DMARD), a biological response modifier, or a corticosteroid. Biological response modifiers include, e.g., an anti-TNF agent (e.g., a soluble TNF receptor or an antibody specific for TNF such as adulimumab, infliximab, or etanercept). The other treatment can also be a physical therapy or physical rehabilitation regimen.

The one or more additional therapeutic agents can also include, e.g., a pain medication (e.g., carbamazepine, gabapentin, topiramate, zonisimide, phenytoin, pentoxifylline, ibuprofen, aspirin, or acetaminophen), an anti-anxiety medication (e.g., fluoxetine, sertraline, vanlafaxine, citalopram, parocetine, trazodone, buproprion, diazepam, or amitriptyline), an incontinence medication (e.g., oxybutynin, bethane, or imipramine), an anti-tremor or spasticity medication (e.g, baclofen, dantrolene sodium, or tizanidine), or an agent that prevents or ameliorates vertigo (e.g., mecizine, dimenhydrinate, prochlorperazine, or scopolamine).

As described in the foregoing sections, the one or more additional therapeutic agents can, optionally, be formulated in the stem cell compositions (where appropriate), or can be administered in conjunction with the compositions as follows.

The stem cells or pharmaceutical compositions and the one or more additional agents can be administered at the same time. Alternatively, the stem cells can be administered first in time and the one or more additional agents administered second in time. The one or more additional agents can be administered first in time and the stem cells administered second in time. The stem cells can replace or augment a previously or currently administered therapy. For example, upon treating a subject with stem cells as described herein, administration of the one or more additional agents can cease or diminish, e.g., be administered at lower levels. Administration of the previous therapy can also be maintained. In some instances, a previous therapy can be maintained until the level of the stem cells (e.g., the amount or schedule) reaches a level sufficient to provide a therapeutic effect. The two therapies can be administered in combination.

In some instances, when the subject is administered stem cells or pharmaceutical compositions, the first therapy is halted. The subject can be monitored for a first pre-selected result, e.g., an improvement in one or more symptoms of an injury to the CNS such as any of those described herein (e.g., see above). In some cases, where the first pre-selected result is observed, treatment with the stem cells is decreased or halted. The subject can then be monitored for a second pre-selected result after treatment with the stem cells is halted, e.g., a worsening of a symptom of an injury to the CNS. When the second pre-selected result is observed, administration of stem cells to the subject can be reinstated or increased, or administration of the first therapy is reinstated, or the subject is administered both stem cells and a first therapy, or an increased amount of the stem cells and the first therapeutic regimen.

The following are examples of the practice of the invention. They are not to be construed as limiting the scope of the invention in any way.

### Examples

### Example 1

### Materials and Methods

Patients Guidelines. All studies were approved in accordance with the ethical committee of Luis Vernaza Hospital, Guayaquil, Ecuador. Patients with acute or chronic spinal cord injuries were enrolled in this study and an informed consent was obtained from each patient. All patients were evaluated prior to enrollment in this study. Some of the inclusion criteria included, e.g.: a spinal cord injury with paraplegia or paraparesia; not having any impediments to effect the analysis of the spinal cord with magnetic resonance imaging; being over the age of 13 with consent of the responsible adult if under 18; having a desire and being motivated to participate in the study; and having an absolute understanding of the informed consent. Some of the exclusion criteria were: doubting an ability to follow the specific study outlined; depression, psychosis or any other mental disorders; alcohol or drug abuse; other diseases especially those with blood related disorders; active infections; patients who have taken immunosuppressants one month prior to the study; multiple acute injuries; active pressure ulcers of the skin especially in the iliac crest region; an inability to follow a strict physical therapy regimen; obesity; and a life expectancy of less than 2 years. Following admittance into the study, patients underwent an extensive medical evaluation including magnetic resonance imaging (MRI), psychological examination, and neurological examination by physicians trained with the Frankel scale and American Spinal Injury Association (ASIA) impairment scale. Patients were also evaluated using the Ashworth scale (spasticity), Barthel Index (quality of life), and a newly developed bladder function scale designated the Geffner, Gonzalez, Santacruz, and Flor (GGSF) Bladder Function Scale. All patients underwent standard physical therapy prior to and after transplantation. Patients were classified into acute or chronic injury according to the International Campaign for Cures of spinal cord injury Paralysis (ICCP) guidelines (chronic injuries are defined as patients who have been injured longer than 1 year where the preceding 6 months there were no changes in functional capacity) (Fawcett et al. (2006) Spinal Cord 45:190-205). All acute patients were not treated with any other medications prior to BMSCs transplantation.

Isolation of Autologous Bone Marrow Stem Cells for Administration. Bone marrow was harvested by aspiration at a minimal number of sites and under intrathecal (or no) anesthesia depending on the individual case. 100 ml of bone marrow was harvested using only one skin puncture site on the right and left sides. A multi-holed needle was introduced into the iliac bone between both posterior iliac spines. 5 ml aspirations were collected at a time for a total of 10 aspirations on the left and 10 aspirations on the right. The bone marrow was placed in a blood collecting bag with 15,000 units of sodium heparin and kept on ice. Using a satellite bag system and centrifugation at 1500 rpm for 20 minutes, the buffy coat layer was separated and obtained. The buffy coat was transferred into a bag containing 75 ml of ficol-hypaque with 5,000 units of heparin and centrifuged at 1000 rpm for 30 minutes. The supernatant, which contained the mononuclear cells, was then washed with sterile saline solution and placed into a blood collecting bag and a sample was processed for FACS analysis to obtain CD34⁺/CD45⁻ cell counts. The mononuclear cells were resuspended in saline and autologous plasma for a total volume of 80 ml. The average total of mononuclear cells obtained for transplantation was 4×10⁸ cells. Within that administration population was an average of 90×10⁶ CD34⁺ cells.

Identification of Cells by Fluorescence Activated Cell Sorting (FACS). FACS analysis was performed using the FACS Calibur from Becton, Dickinson (BD) (Franklin Lakes, N.J.). The ISHAGE method was used for obtaining CD34⁺ cell counts as previously described (Garbossa et al. (2006) Neurol. Res. 28:500-504). Briefly, samples were incubated with monoclonal antibodies specific for CD34 and CD45 followed by Pharm Lyse Lysing solution, TruCount Tubes and Via Probe (7-AAD) (all from BD). The acquisition and analysis of data was performed using Cell Quest software (BD). Prior to data acquisition, a gate was established to exclude CD34⁻, CD45⁻ events in order to eliminate any debris that may contaminate the sample.

Administration of Bone Marrow Stem Cells (BMSC). All patients were administered BMSCs using the same paradigm. Under general anesthesia, a radioscopic assessment of the vertebral injury area was performed. After careful evaluation of the injury site, a laminectomy(s) was performed in order to expose the spinal cord. Following clear visibility of the spinal cord, the scar tissue was carefully removed and the cord detethered. Using a 21 gauge needle attached to a syringe, multiple micropunctures were then performed and 1 ml of cell suspension was injected into multiple locations in and around the injury epicenter and into any intraspinal cavities for a total of 20 ml. The dura was then sutured shut and another 30 ml of the cell suspension was administered into the spinal canal. The remaining 30 ml was intravenously administered for a total of 80 ml of cell suspension.

Neurological Evaluation. Following acceptance into the study, all patients underwent an initial evaluation using the ASIA, Frankel, and Ashworth scales. Follow up testing was done at approximately 6 months, 1 year, and 2 years after administration, except in a few cases. None of the neurologists involved in the study participated the recruitment phase of the study. The ASIA scale was used to evaluate motor and sensory function as previously described (Syková et al. (2006) Cell Transplant. 15:675-87). The Frankel score was also used to classify each SCI patient with definitions as follows: A=complete paralysis; B=sensory function only below the injury level; C=incomplete motor function below injury level; D=fair to good motor function below injury level; and E=normal function. The widely used and accepted modified Ashworth score (Katz et al. (1997) PM&R Secrets. Philadelphia; Hanley & Belfus; 487) was used in order to measure spasticity changes following transplantation with definitions as follows: 0=no increase in tone, 1=slight increase in muscle tone, manifested by a catch and release or minimal resistance at the end of the ROM when the affected part(s) is moved in flexion or extension; 1+slight increase in muscle tone, manifested by a catch, followed by minimal resistance throughout the remainder (less than half) of the ROM; 2=more marked increase in muscle tone through most of the ROM, but affected part(s) easily moved; 3=considerable increase in muscle tone, passive movement difficult; and 4=affected part(s) rigid in flexion or extension. These three clinical parameters were evaluated because they are widely accepted assessment methods used in SCI.

Quality of Life Evaluation and Bladder Function Evaluation. The Barthel Index was used to document changes in quality of life following administration of BMSCs to the patients. Briefly, there are 10 categories (feeding (0, 5, 10), bathing (0, 5), grooming (0, 5), dressing 0, 5, 10), bowels (0, 5, 10), bladder (0, 5, 10), toilet use (0, 5, 10), transfers-bed to chair and back (0, 5, 10, 15), mobility-on level surfaces (0, 5, 10, 15), and stairs (0, 5, 10)) for a maximum score of 100. After carefully evaluating bladder function with ultrasounds of the kidneys, ureters, and bladder, a need for a simplified in depth scoring system was needed, which system takes into account the method of voiding following SCI. The Geffner, Gonzalez, Santacruz, and Flor (GGSF) scale was designed. The GGSF scale is a bladder function scoring system from 0-6, wherein a score of 0 is no urinary bladder sensation or function^{a, b, c, d}; 1 indicates patients with cystostomies that when are closed may involuntarily void through the urethra^{a}; 2 indicates bladder sensation or autonomic symptoms and inability to void^{a, b, c, d}; 3 indicates bladder sensation or autonomic symptoms and passive voiding (spontaneous release of urine)^{a, b, c}; 3.5 indicates patients with open cystostomies that have bladder sensation or autonomic symptoms and passively void through the urethra (spontaneous release of urine)^{a}; 4 indicates bladder sensation with incomplete voiding (needs catheterization to complete voiding)^{b, c, d}; 5 indicates bladder sensation with active ability to void; however no control while voiding; and 6 indicates complete bladder control.
^{a}. Patients use a suprapubic cystostomies in order to void their urinary bladder;
^{b}. Patients use a catheter in order to void their urinary bladder;
^{c}. Patients with a urine collector;
^{d}. Patients that manually compress (massage) the hypogastric region in order to void their urinary bladder.

Magnetic Resonance Imaging (MRI). For all MRI studies a General Electric 1.0 Tesla closed system was used. In order to determine the site of injury, we analyzed several T1 and T2-weighted images as previously described (Flanders et al. (1999) Am. J. Neuroradiol. 20:926-934). Prior to acceptance into the study, patients underwent MRI and only patients with clear midsagittal T2-weighted images of the lesion site were allow into the study. The lesion site was quantified according to the midsagittal T2-weighted image and the vertebral segments were identified in order to isolate the area of interest for administration of BMSCs.

### Example 2

### Study Results

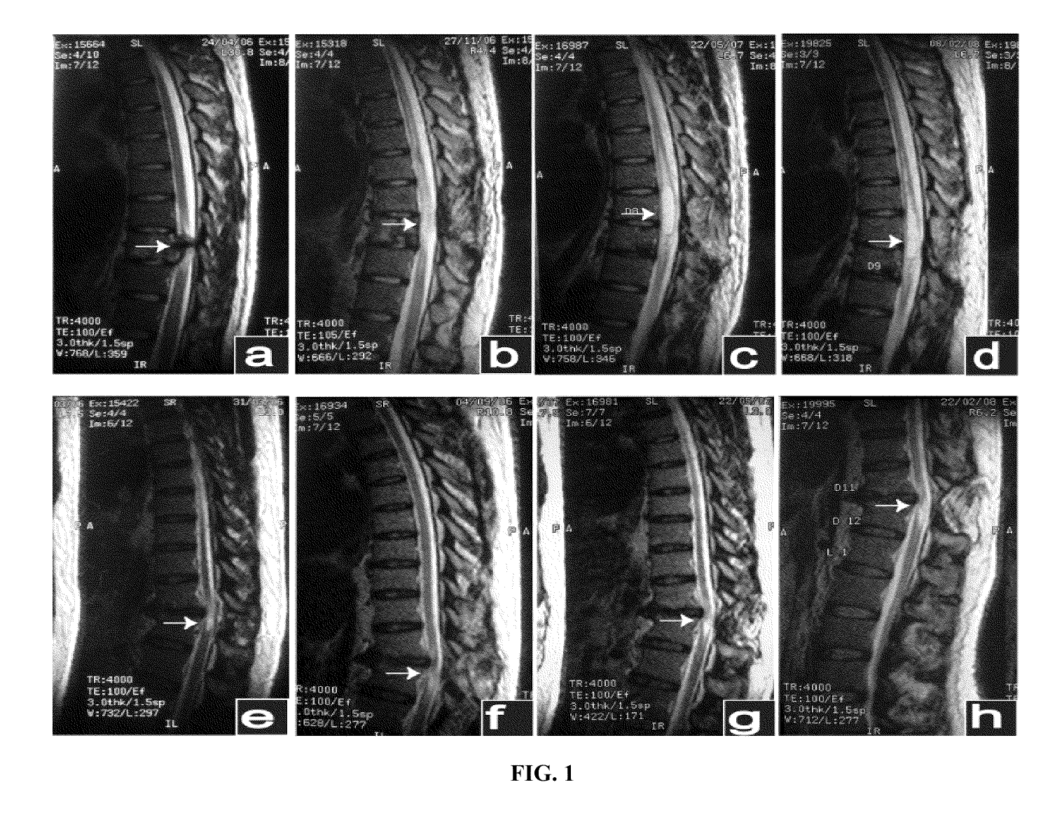

Table 1 illustrates the demographics of each case (cases 1-4 are acute; cases 5-8 are chronic). Bone marrow isolated from each patient was evaluated by FACS analysis for the presence of CD34⁺ stem cells (Table 1). The patients were administered with an average of 1.2×10⁶ CD34⁺ cells per kilogram of body weight for an average total of 90.0×10⁶ CD34⁺ cells per administration (Table 1). Prior to the administration, each patient underwent an MRI and neurological examination (as described above). At approximately 6 months, 1 year, and 2 years following BMSCs administration, the patients all underwent follow up MRIs and neurological exams. Following administration of BMSCs, there were noticeable morphological changes within the spinal cord as illustrated by sequential MRIs of an acute patient (FIGS. 1A-1D) and chronic patient (FIGS. 1E-1H) taken prior to administration, 6 months after BMSCs administration, 1 year after BMSCs administration, and approximately 2 years after BMSCs administration (FIG. 1). These studies indicate that administration of BMSCs (i) directly into the spinal cord, (ii) directly into the spinal canal, and (iii) intravenously cause morphological changes to the spinal cord.

TABLE 1
Case Study Demographics

| | | | | | | | Viability | Time of BMSCs |
|---|---|---|---|---|---|---|---|---|
| | | | Weight | Injury | Injury | CD34/kg | CD34+ | Administration |
| Case # | Sex | Age | (kg) | Level | Type | Cell/10e6 | (%) | After SCI |
| 1 | M | 28 | 80 | T9 | Gunshot | 1.43 | 89.62 | 1.5 Months |
| 2 | F | 33 | 75 | T4 | Gunshot | 1.1 | 82.22 | 7 Months |
| 3 | M | 28 | 79 | T5-6 | Fall | 1.5 | 77.62 | 13 Days |
| 4 | M | 31 | 67 | T12- | Fall | 0.94 | 96.27 | 5 Days |
| | | | | L1 | | | | |
| 5 | M | 37 | 86 | T12 | Car | 1.2 | 91.22 | 6 Years 3 Months |
| | | | | | Accident | | | |
| 6 | M | 42 | 72 | T4 | Gunshot | 1.3 | 91.93 | 21 Years 10 Months |
| 7 | M | 27 | 80 | T11 | Gunshot | 0.88 | 91.15 | 5 Years 10 Months |
| 8 | M | 44 | 68 | T12 | Fall | 1.43 | 89.62 | 6 Years 9 Months |

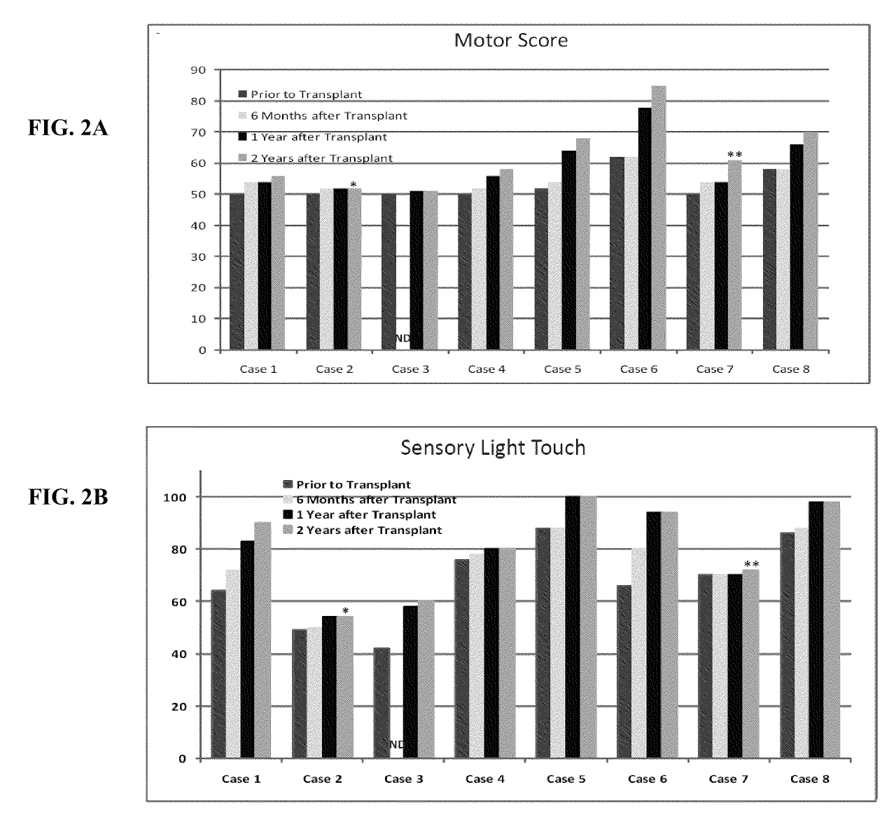

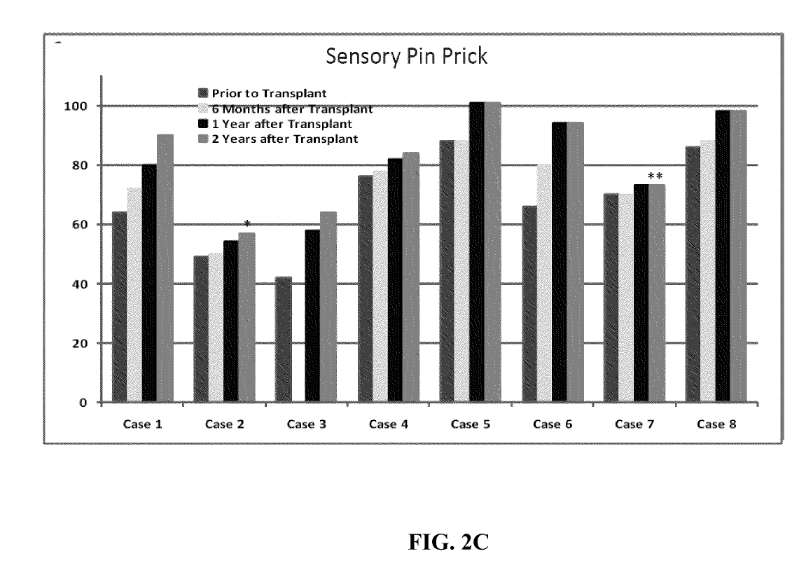

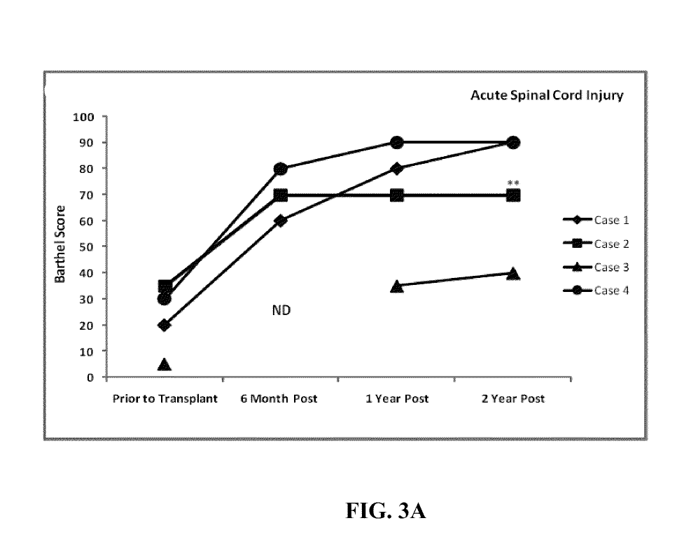

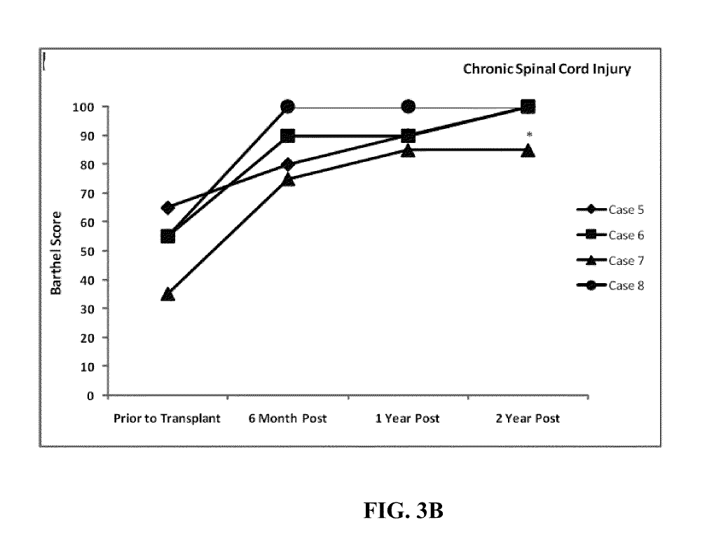

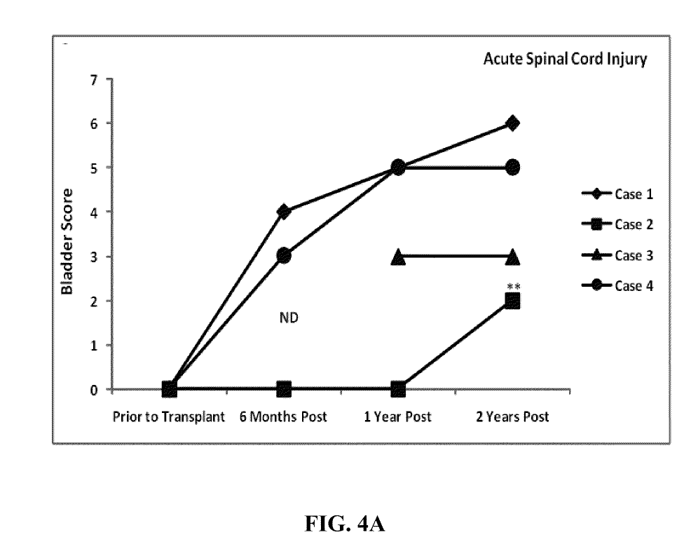

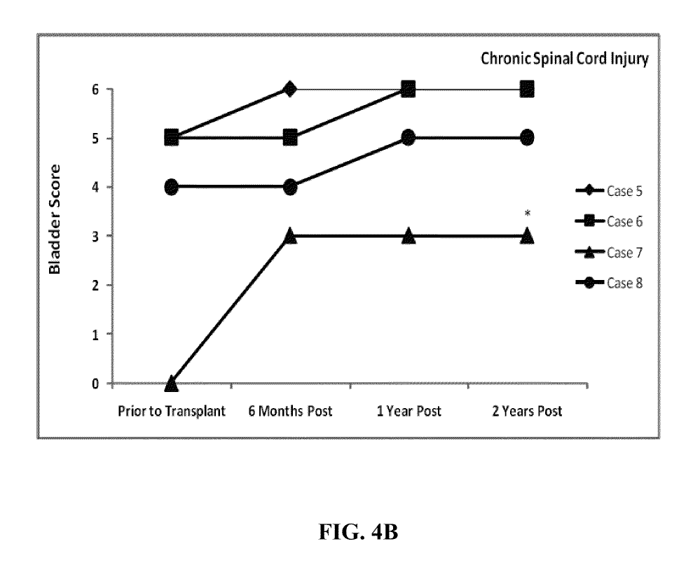

Case 1. A 28 year old male sustained a gunshot wound to the T9 vertebral body resulting in a lesion and contusion injury with a metallic fragment in the spinal canal. Initial evaluation of the patient's MRI illustrated a lateral hemisection of the spinal cord at T9.1-T9.2 resulting from the bullet. In addition, there was a contusion at the T8.1-T10.1 levels with edema and spinal cord thickening (FIG. 1A). The patient's evaluation prior to BMSCs administration demonstrates that he sustained a complete injury (ASIA impairment grade A, motor score 50, Frankel grade B) with no motor functions preserved below the level of injury. Moreover, there was no sensation (light touch or pinprick; score of 64, 64 respectively) below the S2 dermatome (Table 2, Table 3; and FIGS. 2A-2C). Comprehensive data analysis indicates an initial Barthel scoring of 20 (FIG. 3A), Ashworth 0 (Table 2), and bladder function (GGSF) score of 0 (FIG. 4F). Forty days post injury, the patient had the bullet removed and was administered BMSCs. MRI of the vertebral column approximately two years after administration of the BMSCs illustrated the continual existence of the lateral hemisection with residual cavities formed from T7.1-T9.1 and spinal cord thickening (FIG. 1D). In addition, there appeared to be the formation of a small bridge of tissue with the same MRI intensity of normal tissue and dorsal recuperation of normal spinal cord signal below the injury level. Approximately two years post BMSCs, administration the patient had progressed significantly with an ASIA impairment grade of C, motor score 56, and Frankel grade of C. In addition, there was sensation through the S4-5 dermatome (light touch or pinprick; score of 90, 90, respectively) (Table 2, Table 3, and FIGS. 2A-2C). Quality of life (Barthel score) increased from a score of 20 to 90 (FIG. 3A) and bladder function improved from no function to complete bladder control (FIG. 4A). The patient could stand on parallel bars and take small strides with the use of a walker or crutches. The patient came to physical therapy riding a quad motor bike.

TABLE 2
Neurological Evaluations

| | | | | |
|---|---|---|---|---|
| | | | | 2 Years after |
| Case | Prior to | 6 Months after | 1 Year after | Admin- |
| Study | Administration | Administration | Administration | istration |

ASIA IMPAIRMENT GRADE/
FRANKEL GRADE/ASHWORTH SCORE

| | | | | |
|---|---|---|---|---|
| Acute | | | | |
| Case 1 | A/B/0 | C/C/2 | C/C/3 | C/C/1 |
| Case 2 | A/A/3 | A/C/1 | A/C/1 | C/C/2** |
| Case 3 | A/A/0 | ND | A/C/1 | A/C/1 |
| Case 4 | A/A/0 | C/C/1 | C/C/1 | C/C/1 |
| Chronic | | | | |
| Case 5 | B/C/1 | B/C/0 | C/D/1 | C/D/1 |
| Case 6 | C/D/3.5 | C/D/3 | D/D/3.5 | D/D/ND |
| Case 7 | A/A/0 | C/C/1 | C/C/1 | C/C/1+* |
| Case 8 | C/C/2 | C/C/2 | C/D/1 | C/D/0 |

ND—Not done
*1 year 3 months
**1 year 6 months

TABLE 3
ASIA Motor and Sensory Scores

| | | | | |
|---|---|---|---|---|
| | | | | 2 Years after |
| Case | Prior to | 6 Months after | 1 Year after | Admin- |
| Study | Administration | Administration | Administration | istration |

ASIA Motor Score/Sensory Light Touch Score/Sensory Pin Prick Score

| | | | | |
|---|---|---|---|---|
| Acute | | | | |
| Case 1 | 50/64/64 | 54/72/72 | 54/83/80 | 56/90/90 |
| Case 2 | 50/49/49 | 52/50/50 | 52/54/54 | 52/54/57* |
| Case 3 | 50/42/42 | ND | 51/58/58 | 51/60/64 |
| Case 4 | 50/76/76 | 52/78/78 | 56/80/82 | 58/80/84 |
| Chronic | | | | |
| Case 5 | 52/88/88 | 54/88/88 | 64/100/101 | 68/100/101 |
| Case 6 | 62/66/66 | 62/80/80 | 78/94/94 | 85/94/94 |
| Case 7 | 50/70/70 | 54/70/70 | 54/70/73 | 61/72/73** |
| Case 8 | 58/86/86 | 58/88/88 | 66/98/98 | 70/98/98 |

ND—Not done
*1 year 3 months
**1 year 6 months

Case 2. A 33 year old female sustained a gunshot wound to the thoracic area resulting in an injury to the spinal cord at the T4 vertebral level. Initial evaluation of the patient's MRI illustrated a transection at T5.2-T6.1 and a severe contusion injury with edema from T4.3 to T5.1. The patient's evaluation prior to BMSCs administration demonstrated that she sustained a complete injury (ASIA impairment grade A, motor score 50, Frankel grade A) with no motor functions preserved below the level of injury. In addition, there was no sensation (light touch or pinprick; score of 47, 47 respectively) below the T7 dermatome (Tables 2 and 3 and FIGS. 2A-2C). Eleven days following her SCI, the patient underwent a first surgery in order to decompress the spinal cord and remove the bullet fragment from the spinal canal. Following decompression, the patient had no functional changes even though she underwent a strict rehabilitation regimen. Administration of BMSCs was performed 7 months after her spinal cord injury. At 6 months following BMSCs administration, the patient progressively started to improve (Tables 2 and 3 and FIGS. 2A-2C, 3A, and 4A). At 6 months post administration, her ASIA motor and sensory scoring had improved slightly (52, 50, and 50 for motor, sensory light touch, and sensory pin prick respectively) (Table 3; FIG. 2A-2C), while her quality of life (Barthel Score) improved 35 points (FIG. 3A). Her most recent MRI (18 months) illustrated the persistence of a complete transection at T5.2-T6.1 with scar tissue at T5.2-T6.1. In addition, there was retraction of supra-posterior cord fibers. There was no evidence of edema. At 18 months following BMSCs administration, her bladder function improved from none to having sensation (FIG. 4A). Also, she improved from an ASIA impairment grade A to C (52, 54, and 57 for motor, sensory light touch, and sensory pinprick respectively), Frankel A to C, and her spasticity had decreased from a 3 to 2 (Tables 2 and 3 and FIGS. 2A-2C). There was no sensation below the T9 dermatome. Overall, a qualitative increase in her quality of life was observed. She can now stand on the parallels with braces.

Case 3. A 28 year old male fell from a tree approximately eight meters and sustained an injury to his spinal cord at the T5-6 vertebral spinal level. Initial MRI illustrated an oblique hemisection on the left side at T5.1-T6.1 and contusion with edema cranial at T4.3-T3.1 and caudal at T6.2-T7.3. There was also dilation of the ependyma below the injury and posterior vertebral displacement narrowing the canal. Initial neurological evaluation demonstrated that the patient sustained a complete injury (ASIA impairment grade A, Frankel A) with no motor function preserved below the injury area (motor score 50) (Table 2 and 3 and FIGS. 2A-2C). In addition, there was no sensation (light touch or pinprick; score of 42, 42 respectively) below the T5 dermatome (Table 3 and FIGS. 2B and 2C). The patient was administered BMSCs 13 days following his SCI and metal rods were placed in order to stabilize his vertebral column. The patient never showed up for his rehabilitation regimen and was given inadequate care at home immobile for 32 days. The patient acquired a giant bed sore that had to be surgically removed. The bed sore caused an infection leading to a definitive colostomy and removal of the left femoral head. Due to the severe bed sore, the patient had many autologous skin graft surgeries in order to repair the damaged area. The patient had an initial Barthel score of 5 (FIG. 3A), bladder function score of 0 (FIG. 4A), and Ashworth (spasticity) score of 0 (Table 2). At approximately 2 years post BMSCs administration, the patient had an improved Frankel grade of C, an ASIA impairment grade of A with some improvements in his sensory score (60, 64 for light touch and pin prick respectively) and no sensation below the T11 dermatome (Table 2 and 3 and FIGS. 2B and 2C). His quality of life score (Barthel Index) had increased from 5 to 40 (FIG. 3A) and bladder function increased from no bladder function to bladder sensation or autonomic symptoms and passive voiding (spontaneous release of urine) (FIG. 4A). Additional MRIs were not performed after placement of the metal rods. Notwithstanding the challenges presented with his bed sore and the fact that he has not performed any rehabilitation regimen, this patient's quality of life definitely improved following administration of BMSCs.

Case 4. A 31 year old male fell from a ladder approximately three meters and sustained an injury to his spinal cord at the T12-L1 vertebral level. The patient was evaluated immediately after injury. MRI of the vertebral column illustrated posterior vertebral displacement of L1 over T12 grade II and severe narrowing of the spinal canal. There was a contusion with edema at T12.2-L1.3 with a hematoma. The initial neurological evaluation demonstrates that the patient sustained a complete injury (ASIA impairment grade A, Frankel A) with no motor function preserved below the injury level (motor score 50) (Table 2 and 3 and FIG. 2A). There was no sensation (light touch or pinprick; score of 76 and 76 respectively) below the T12 dermatome (Table 3; FIGS. 2B and 2C) and he had an initial Ashworth score of 0 (Table 2). Quality of life scoring demonstrated an initial Barthel score of 30 (FIG. 3A) and no bladder function (FIG. 4A). Five days following initial trauma, posterior rods were placed in order to stabilize the vertebral column and administration of BMSCs was done. At two years following BMSCs administration, the patient had improved significantly (ASIA impairment grade C, motor score 58, Frankel score C) with active movement against gravity at the hip flexors and sensation through the L1 dermatome and at the S2-S3 dermatome (light touch and pin prick; 84 and 80, respectively) (Tables 2 and 3 and FIGS. 2A-2C). Barthel scoring demonstrated an improvement from an initial score of 30 to 90 (FIG. 3A), while bladder function went from absolutely no function to bladder sensation with active ability to void. However, the patient exhibited no control while voiding (FIG. 4A). The metal rods prevented subsequent MRI. This patient could walk a couple of blocks with the use of a walker and ankle braces.

Case 5. A 37 year old male sustained an injury to the spinal cord at the T12 vertebral spinal level from a car accident. The patient was evaluated 6 years 2 months following injury. MRI prior to BMSCs administration illustrated an anterior disc herniation at T11-12 and a lateral hemisection of the spinal cord at T11.3. In addition, there was a residual cavity from T12.1 to T12.2 (FIG. 1E). The initial neurological evaluation demonstrated that the patient sustained an incomplete injury (ASIA impairment grade B, Frankel C) with only palpable or visible contractions at the L2 level (ASIA motor score 52, 1 point on each side at the L2 level) (Tables 2 and 3 and FIG. 2A). In addition, there was impaired sensation (light touch or pinprick; score of 88 and 88, respectively) through the S4-5 dermatome (Table 3 and FIGS. 2B and 2C) and an Ashworth score of 1 (Table 2). Initial Barthel score was a 65 (FIG. 3B) and he had bladder sensation with active ability to void. However, the patient had no control while voiding (GGSF score of 5) (FIG. 4B). 6 years and 3 months following his SCI, the patient underwent a partial discectomy and administration of BMSCs. At 2 years following administration of the BMSCs, the patient had significantly improved with the MRI illustrating a disc herniation at T11-12, a lesion at T11.3, and a small cavity at T12.1 (FIG. 1H). His neurological evaluation demonstrated an ASIA impairment grade of C (improved motor score of 68, scores of 1-3 from the L2-S1 level) and Frankel D (Tables 2 and 3 and FIG. 2A). His ASIA sensory score had increased from 88 light touch and 88 pin prick to 100 light touch and 101 pin prick (Table 3 and FIGS. 2B and 2C). The Barthel score for this patient elevated to 100 (maximum value) and he had regained full control of his bladder (FIGS. 3B and 4B, respectively). This patient regained the ability to walk with braces and crutches for more than one hour.

Case 6. A 42 year old male sustained a gunshot wound to the chest in 1984 which penetrated his vertebral column and was lodged in his dura at the T4 vertebral spinal level causing an injury to his spinal cord. The patient immediately underwent a laminectomy at T3-4 and surgical removal of the bullet. An MRI performed prior to BMSCs administration showed that the patient had an oblique lesion at T3.2-T4.1 and hypoplasia. There was also a residual cavity at T3.2-T4.2. A neurological evaluation prior to administration (21 years 10 months after initial trauma) demonstrated that he was categorized as an incomplete injury (ASIA impairment grade C, Frankel D) with active movement, gravity eliminated through the L4 level on the left/L3 level on the right and palpable or visible contractions at the L5-S1 level on the left side only (ASIA motor score 62) (Table 2 and 3 and FIG. 2A). In addition, there was no sensation below the T7 dermatome on the right and impaired through the S4-5 dermatome from T6 (light touch or pinprick; score of 66 and 66 respectively) (Table 3 and FIGS. 2B and 2C). The initial Barthel score was 55 (FIG. 3B) and the patient had bladder sensation with active ability to void (but with no control while voiding; score of 5) (FIG. 4B). Minor changes with an oblique lesion at T3.3-T4.3 and hypoplasia were observed by MRI at the patient's 2 year follow-up. The residual cavity at T3.2-T4.2 also remained. A neurological evaluation at 2 years post BMSCs administration demonstrated a significant improvement of motor function with an ASIA impairment grade of D, motor score 94, and Frankel D grade (Table 2 and 3 and FIG. 2A). Moreover, his sensory scoring had improved from a 66 light touch/66 pin prick to a 94 light touch/94 pin prick (Table 3 and FIGS. 2B and 2C). The patient's Barthel score had improved dramatically to a maximum score of 100 and he has complete bladder function (FIGS. 3B and 4B, respectively). The patient regained the ability to walk using forearm crutches and the ability to walk up and down stairs.

Case 7. A 27 year old male sustained a gunshot wound to the scapula region which passed through his spine and exited his chest causing a spinal cord injury at the T11 vertebral level. The patient was evaluated for BMSCs administration 5 years 9 months follow SCI. Initial MRI illustrated a fracture at T11.1 with a cavity from the projectile in the vertebral body and an oblique hemisection of the spinal cord at T11.1-T11.2 His neurological evaluation prior to BMSCs administration demonstrated that he has an complete injury (ASIA impairment grade A, Frankel A) with a motor score of 50 meaning the absence of all key muscles below the injury level (Table 2 and 3 and FIG. 2A). There was no sensation below the T11 dermatome (70 and 70 for light touch and pin prick respectively) and his Ashworth score was 0 (Table 2 and 3 and FIGS. 2B and 2C). His Barthel score was 35 and he had no bladder function (FIGS. 3B and 4B, respectively). MRI evaluation 1 year 3 months following BMSCs administration illustrated the existence of the fracture at T11.1 and the oblique hemisection injury had expanded from T11.1-T12.1. There was a small residual cavity above the injury from T10.1-T10.3. Although his MRI demonstrates an expanded injury, his neurological evaluation indicated a significant functional improvement (ASIA impairment grade C, improved motor score of 61) (Table 2 and 3 and FIG. 2A). In addition, his sensory score has increased slightly with impaired sensation at the S2-S3 dermatome level (light touch 72, pin prick 73) (Table 3 and FIGS. 2B and 2C). There were little changes in his spasticity. Barthel scoring indicated a significantly improvement to a score of 85 (FIG. 3B) and his bladder function has improved from 0 to a 3 (sensation or autonomic symptoms and passive voiding) (FIG. 4B). One year 3 months following the administration of BMSCs, the patient regained the ability to walk with the aid of a walker and braces.

Case 8. In 1999, a 44 year old male fell 8 meters and sustained an injury to the spinal cord at the T12 vertebral spinal level. Five days after his SCI, the patient had metal rods placed in order to stabilize his vertebral column, which were subsequently removed 4.5 years later. The patient was evaluated at the hospital for BMSCs administration in early 2006. His MRI prior to BMSCs administration showed a compression of the vertebral body at T12 with a 10% posterior displacement. There was compression of the spinal cord at T11.2-T12.1 with a residual cavity at T11.2-T12.3. His initial neurological examination with us indicated that he had an incomplete injury (ASIA impairment grade C, Frankel C) with active movement, gravity eliminated at L2 and L3 (motor score 58) (Table 2 and 3 and FIG. 2A). There was no sensation below the L3 dermatome (86 and 86, light touch and pin prick respectively) and his Ashworth score was 2 (Table 2 and 3 and FIGS. 2B and 2C). His Barthel score was a 55 (FIG. 3B) and he had bladder sensation with incomplete voiding (score of 4) (FIG. 4B). Two years following the administration of BMSCs, the patient's MRI identified a lesion at T11.3-T12.1 with a residual cavity which has shrunk at T11.2-T11.3. There was also a small edema below the lesion at T12.2-T12.3. The neurological evaluation indicated functional improvement (ASIA C, improved motor score of 70, and Frankel D) and a reduction in spasticity (Table 2 and 3 and FIG. 2A). There was impaired sensation through the S4-5 dermatome with an improved sensory score of 98 and 98 for light touch and pin prick respectively (Table 3 and FIGS. 2B and 2C). The Barthel scoring indicated that the patient had improved to the maximum score of 100 (FIG. 3B) and had the ability to actively void his bladder (GGSF score 5) (FIG. 4B). Even though the patient had the ability to walk with crutches and can go up and down stairs, since he has active movement against full resistance (score 5) in only 40% of his key muscles, he continues to be considered at ASIA impairment grade C.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What we claim is:

1. A method for treating an injury to the central nervous system, the method comprising delivering a therapeutically effective amount of stem cells to a subject having an injury to the central nervous system, wherein the delivering comprises:
administering a first composition comprising stem cells locally to the epicenter of the injury; and
administering a second composition comprising stem cells intravenously.

2. The method of claim 1, wherein administering the first composition comprises two or more injections to the epicenter of the injury.

3. The method of claim 2, wherein at least one of the two or more injections is into the grey matter of the epicenter.

4. The method of claim 1, further comprising removing scar tissue from the site of injury prior to delivering the stem cells.

5. The method of claim 4, wherein the scar tissue comprises a glial scar.

6. The method of claim 1, wherein the injury is a brain injury.

7. The method of claim 1, wherein the injury is a spinal cord injury.

8. The method of claim 7, further comprising detethering the spinal cord.

9. The method of claim 7, further comprising performing a laminectomy on the subject.

10. The method of claim 1, wherein the delivering further comprises administering a third composition comprising stem cells into the subarachnoid space or the spinal canal of the subject.

11. The method of claim 1, wherein the stem cells delivered to the subject are autologous stem cells.

12. The method of claim 1, further comprising, prior to the delivering, culturing at least a portion of the stem cells under conditions that permit differentiation of the cells.

13. The method of claim 12, wherein the conditions permit differentiation of a plurality of the stem cells into neuronal cells.

14. The method of claim 1, wherein the injury results from physical trauma, a cancer, an ischemic event, a developmental disorder, a neurodegenerative disorder, an inflammatory disorder, or a vascular malformation.

15. The method of claim 1, wherein the injury is an acute injury.

16. The method of claim 1, wherein the injury is a chronic injury.

17. The method of claim 1, wherein the stem cells delivered to the subject comprise CD34⁺ bone marrow-derived stem cells.

18. The method of claim 1, wherein the stem cells delivered to the subject comprise embryonic stem cells.

19. The method of claim 1, wherein the stem cells delivered to the subject comprise adult stem cells.

20. The method of claim 1, wherein at least 1 million stem cells/kg mass of the subject are delivered to the subject.

21. The method of claim 1, further comprising subjecting the subject to a physical therapy regimen after delivering the stem cells.

22. The method of claim 1, wherein the subject is a human.

23. The method of claim 1, wherein the subject is a human, the injury is a spinal cord injury, and the delivering comprises:
administering a first composition comprising CD34⁺ bone marrow-derived stem cells locally to the epicenter of the spinal cord injury;
administering a second composition comprising CD34⁺ bone marrow-derived stem cells intravenously; and
administering a third composition comprising CD34⁺ bone marrow-derived stem cells into the subarachnoid space.

24. The method of claim 1, wherein the subject is a human, the injury is a spinal cord injury, and the method comprises:
removing scar tissue from the injured spinal cord;
detethering the spinal cord;
administering a first composition comprising CD34⁺ bone marrow-derived stem cells locally to the epicenter of the spinal cord injury;
administering a second composition comprising CD34⁺ bone marrow-derived stem cells intravenously; and
administering a third composition comprising CD34⁺ bone marrow-derived stem cells into the subarachnoid space.
